(19) United States
(12) Patent Application Publication
(10) Pub. No.: US 2009/0105348 A1
(43) Pub. Date: Apr. 23, 2009

(54) TREATMENT OF MIGRAINE HEADACHES USING ANTIESTROGENS

(76) Inventor: HARRY FISCH, Scaresdale, NY (US)

Correspondence Address: NORRIS, MCLAUGHLIN & MARCUS, PA, 875 THIRD AVENUE, 18TH FLOOR, NEW YORK, NY, 10022, US

(21) Appl. No.: 12/253,431

(22) Filed: Oct. 17, 2008

Related U.S. Application Data
(60) Provisional application No. 60/980,909, filed on Oct. 18, 2007.

(51) Int. Cl. A61K 31/138; A61P 25/06

(52) U.S. Cl. 514/648

(57) ABSTRACT

A method of preventing the occurrence of migraine headaches in a patient who is a previous sufferer of migraine headaches, especially accompanied by prodrome and/or aura, comprises administering an effective amount therefor of an antiestrogen to the patient for a period of time and sufficiently far in advance of the occurrence of a migraine headache to prevent the development of the prodromal phase and/or aura phase of the migraine headache.

### PRIORITY CLAIM

This application claims priority of U.S. Provisional Application No. 60/980,909, filed on Oct. 18, 2007.

### BACKGROUND OF THE INVENTION

1. Field of the Invention

The present invention relates to a method of treating people who suffer from migraine headaches.

2. Description of Related Art

Migraines produce intense headache comparable to that of a brain aneurysm rupture. As many as 15% of all people suffer from migraines. In the United States alone, the costs associated with treating migraines and the time at work lost by migraine sufferers amount to billions of dollars on an annual basis.

Migraines have at least three distinct phases, although not all migraine suffers experience all of the phases.

The first stage, which is called the “prodrome phase,” is experienced by 60% of migraine sufferers. The prodrome is characterized by a change in mood, energy levels or passive functions, and can occur for hours before the actual onset of the headache. The mood changes include euphoria, loquaciousness, unprovoked apathy, depression, inertia, drowsiness, irritability, repetitive yawning, aggression and sound sensitivity (phonophobia). These mood changes may be accompanied by nausea and vomiting, as well as paresthesias in the extremities.

The second stage, which is called the “aura phase,” is characterized by fear of light (photophobia) and visual disturbances. The most prevalent form of migraine, so-called “common migraine,” occurs without an aura. However, about 20% of migraine sufferers experience so-called “classic migraine,” which is migraine with aura. In migraine with aura, neurologic symptoms (the aura) usually develop over 5 to 20 minutes and last less than an hour. The most common aura is flashing lights in a herringbone pattern. Some people see bright lights in other geometric patterns, or half of their visual field is blank. Others may experience difficulty speaking, weakness on one side of the body, or numbness or tingling in a hand or arm or on one side of the face.

The third stage, which is called the “headache phase,” is characterized by intense local pain. The typical migraine headache is throbbing, with pain starting on one side of the head and then spreading to both sides. Jabs and jolts of sharp, shooting pain in various areas of the head are common. The onset is gradual, with the pain increasing in intensity for the first 30 minutes to 2 hours, then leveling off and slowly subsiding. The average duration of the headache phase is a day, but it can last for up to 3 days. In 90% of migraine sufferers, the headache is accompanied by nausea, vomiting, or loss of appetite. Other accompanying symptoms include blurred vision, nasal stuffiness, diarrhea, neck stiffness, memory impairment, and difficulty concentrating.

Migraines develop suddenly, and reach maximal intensity very quickly. People who suffer from migraines on a regular basis develop an apprehension and fear of the pain that will ensue from an impending migraine, particularly if they typically experience prodrome and/or aura before the headache. They hyperventilate and tense their neck muscles, which can lead to a concomitant tension headache.

There have been many attempts to treat migraines, but effective treatments remain elusive. Most treatments, unfortunately, provide relief only after the headache phase has begun. On the other hand, U.S. Pat. No. 5,250,529, the entire contents of which are hereby incorporated herein by reference, describes a method of alleviating migraines by administering an effective amount of a mast cell degranulation blocking agent just prior to or during the prodromal phase. Those compounds that are identified as being suitable mast cell degranulation blocking agents include antiestrogens, for example, clomiphene and tamoxifen. According to the teachings of this patent, the release of vasoactive and nociceptive compounds are involved in the precipitation of the migraine and, therefore, administering a mast cell degranulation blocking agent just prior to or during the prodromal phase is effective to alleviate the impending migraine. The patent does not provide a definition for “just prior” or otherwise describe exactly how far in advance of the onset of the prodromal phase the mast cell degranulation blocking agent must be administered in order to provide relief from the impending migraine. Indeed, the specific teachings and working examples relate to situations in which the prodromal phase has already begun.

In any case, one problem with this approach in general is that administering the mast cell degranulation blocking agent just prior to or during the prodromal phase does not avoid the development of the undesirable mood changes and other symptoms that are characteristics of the prodromal phase itself. Indeed, treatment Examples 3 and 4 therein describe how in each case female patients took a mast cell degranulation blocking agent during the prodromal phase and “the migraine failed to appear as determined by the absence of severe headache [i.e., the migraine did not progress to the headache phase] and the disappearance of photophobia and phonophobia [i.e., the prodromal and aura phases had both begun, but subsided upon taking the mast cell degranulation blocking agent.]” In other words, administering the mast cell degranulation blocking agent during the prodromal phase only completely blocked the development of the final headache phase, whereas both the prodromal phase and the aura phase still progressed to a significant extent.

A second problem is, as noted above, that people who suffer migraines often recognize the progression to the headache phase early in the process and become apprehensive and fearful of the impending pain, and, as a result, their actions often cause tension headaches to develop as a complication. Indeed, the patients in the treatment examples of the patent mentioned above were “occasionally” (Example 3) or “infrequently” (Example 4) left with “a dull ache (residual muscle tension headache) which was well tolerated.” In other words, even with the successful blocking of the headache phase, the progression through the prodromal and/or aura phases was enough to trigger in these patients an apprehension and fear of the impending headache sufficient to cause the patients to develop the concomitant tension headache, albeit “well tolerated.” These “residual” difficulties only partly underscore the continuing need in the art to develop treatments that avoid entry into the prodromal and/or aura phases altogether.

In a Letter to the Editor, appearing in Headache: The Journal of Head and Face Pain, Volume 32, page 315 (1992), Dr. Richard Newman reports that one man presenting with headaches and diagnosed as suffering common migraine benefited from taking clomiphene. The letter does not mention that the man experienced a prodrome, but clearly states that the man denied experiencing an aura. Moreover, the headache was accompanied by fever, which is not normally associated with migraine, and, therefore, suggests that migraine may have been misdiagnosed. Further, neurologic examination was normal, whereas at least migraine with aura is now thought to be a neurologic disorder rather than a vascular disorder. See, Hadjikhani N., et al., “Mechanisms of migraine aura revealed by functional MRI in human visual cortex,” PNAS, 98: 4687-4692 (2001); and Lauritzen M., “Pathophysiology of the migraine aura: The spreading depression theory,” Brain, 117: 199-210 (1994).

Accordingly, there remains a need in the art to discover successful treatments for migraine, particularly those that prevent the development of prodrome and/or aura, and also for other headaches.

### SUMMARY OF THE INVENTION

These and other objects were met with the present invention, which relates in a first embodiment to a method of preventing the occurrence of migraine headaches in a patient who is a previous sufferer of migraine headaches, wherein the method comprises administering an effective amount therefor of an antiestrogen to the patient for a period of time and sufficiently far in advance of the occurrence of a migraine headache to prevent the development of the prodromal phase and/or the aura phase of the migraine headache.

The present invention relates in a second embodiment to a method of preventing the onset of the prodromal phase and/or the aura phase of a migraine headache in a patient susceptible to suffering migraine headaches, wherein the method comprises administering an effective amount therefor of an antiestrogen to the patient for a period of time and sufficiently far in advance of the onset of the prodromal phase and/or aura phase to prevent the onset of the prodromal phase and/or aura phase from occurring.

### DETAILED DESCRIPTION OF THE INVENTION

According to the present invention, patients who suffer from migraine headaches can find relief therefrom by taking an effective amount of an antiestrogen for a period of time and sufficiently far in advance of the onset of the prodromal phase and/or aura phase so as to prevent the prodromal phase and/or aura phase from developing in the first place.

The term “patient” as used herein means preferably a human being. In one embodiment, the patient is a man, especially a man having androgen deficiency and/or male menopause, whether the androgen deficiency and/or male menopause results naturally due to advancing age or from androgen deprivation therapy, for example, incident to a treatment for prostate cancer. In one especially preferred embodiment, the patient is a young man between the ages of 20-30 who is androgen deficient. In a second especially preferred embodiment, the patient is an older man at least 50 years of age who exhibits at least one symptom due to androgen deficiency and/or male menopause. In a third especially preferred embodiment, the patient is a man undergoing androgen deprivation therapy, for example, incident to a treatment for prostate cancer. See, for example, U.S. Pat. No. 6,391,920 and U.S. Pat. No. 7,067,557, the entire contents of which are hereby incorporated herein by reference, for symptoms associated with androgen deficiency and/or male menopause and further teachings regarding androgen deficiency and/or male menopause.

In one preferred embodiment, the patient is a migraine sufferer who has experienced migraine headaches involving a prodromal phase, and the inventive treatment prevents the development of the prodromal phase.

In another preferred embodiment, the patient is a migraine sufferer who has experienced migraine headaches involving an aura phase, and the inventive treatment prevents the development of the aura phase.

In an especially preferred embodiment, the patient is a migraine sufferer who has experienced migraine headaches involving both a prodromal phase and an aura phase, and the inventive treatment prevents the development of both the prodromal phase and the aura phase.

The term “antiestrogen” as used herein means any compound that competes with estrogen for estrogen-receptor-binding sites and may delay replenishment of intracellular estrogen receptors. In a preferred embodiment, the antiestrogen is a selective estrogen receptor modulator (SERM) and/or its analog, derivative, isomer, metabolite, pharmaceutically acceptable salt, pharmaceutical product, hydrate, N-oxide, or any combination thereof. In an especially preferred embodiment, the SERMs that are encompassed by the present invention include, but are not limited to the following embodiments: triphenylalkylenes such as triphenylethylenes, which include tamoxifen, droloxifene, toremifene, fispemifene, ospemifene, idoxifene, clomiphene, enclomiphene and zuclomiphene; benzothiphene derivatives such as raloxifene and LY 353381; benzopyran derivatives such as EM 800 (SCH 57050) and its metabolite EM 652; naphthalene derivatives such as lasofoxifene (CP 336,156); chromans such as levormeloxifene or their analogs, derivatives, isomers, or metabolites thereof, or their pharmaceutically acceptable salts, esters, N-oxides, or mixtures thereof.

The term “pharmaceutically acceptable salt” as used herein means pharmaceutically acceptable acidic salts of the free base compound formed, where applicable, with inorganic and/or organic acids, as well as pharmaceutically acceptable basic salts of the free base compound formed, where applicable, with inorganic and/or organic bases. Such pharmaceutically acceptable salts can be formed, for example, by reacting the free base compound with an amount of acid or base, such as an equivalent amount, in a medium such as one in which the salt precipitates or in an aqueous medium followed by lyophilization. Exemplary “pharmaceutically acceptable salts” include, where applicable, and without limitation, alkali metal or alkaline earth metal salts, for example, sodium, potassium, calcium, magnesium or ammonium salts and the like, as well as acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like.

As the antiestrogen, particular preference is given to the use of tamoxifen, tamoxifen citrate, clomiphene, clomiphene citrate or toremifene, especially clomiphene citrate sold under the trademark CLOMID®. CLOMID® (clomiphene citrate) is a mixture of two geometric isomers [cis (zuclomiphene) and trans (enclomiphene)] containing between 30% and 50% of the cis-isomer. Another useful composition is clomiphene citrate wherein the content of the cis isomer ranges from 0-29% by weight of the total trans+cis isomer content in the composition and the content of the trans isomer ranges from 71-100% by weight of the total trans+cis isomer content of the composition. These isomers may be separated and used completely free or substantially free (<10% by weight of the composition) of one another. In a particularly preferred embodiment, the composition contains clomiphene citrate trans isomer completely free or substantially free (<10% by weight of the composition) of cis isomer.

The term “effective amount” as used herein means generally 5 to 1000 mg, preferably 10 to 100 mg, of the antiestrogen, when administered daily or every other day to avert the occurrence of migraines, particularly the onset of the prodromal phase and/or aura phase.

The antiestrogens can be administered, e.g., orally, parenterally or transdermally by a patch or by any other suitable route. Preferably, the antiestrogens are administered orally.

For the preferred oral administration route, suitable means are especially tablets, coated tablets, capsules, pills, suspensions, or solutions that can be produced in a way that is commonly used and familiar to persons skilled in the art, with the additives and vehicles that are commonly used for the formulation of antiestrogens that are to be administered orally.

Further exemplary formulation and administration details can be found in the above-identified patents that have already been incorporated by reference in their entireties.

The pharmaceutical agent that is produced according to the invention contains as an active ingredient per dosage unit of the antiestrogen at a daily or every other day dosage of 5 to 100 mg in addition to the commonly used additives, vehicles and/or diluents or other antiestrogens at biologically equieffective dosages.

For 10 mg tablets, for example, each tablet contains 15.2 mg of tamoxifen citrate which is equivalent to 10 mg of tamoxifen. For 20 mg tablets, each tablet contains 30.4 mg of tamoxifen citrate which is equivalent to 20 mg of tamoxifen. The inactive ingredients are carboxymethylcellulose calcium, magnesium stearate, mannitol and starch.

Commercially available clomiphene citrate tablets typically contain a mixture of two geometric isomers [cis (zuclomiphene) and trans (enclomiphene)] containing between 30% and 50% of the cis-isomer. A standard commercially available tablet contains 50 mg clomiphene citrate and the following inactive ingredients: corn starch, lactose, magnesium stearate, pregelatinized corn starch, and sucrose. The current tablets are used primarily for treating female infertility. Treatment according to the present invention contemplates a redosing to accommodate the lower dosages specified herein.

It is also contemplated that combinations of antiestrogens can be administered.

The term “for a period of time” means at least once daily for a period of at least two days. Preferably, the antiestrogen is administered at least once daily for a period of at least one week. In the most preferred embodiments, the antiestrogen is administered at least once daily for a period of at least one month, or at least one year or continuously for the remainder of the patient's life. For periods of time greater than two days, the antiestrogen may be taken every other day providing this regiment is effective to prevent the start of the prodromal phase and/or aura phase.

The term “sufficiently far in advance” means the antiestrogen is administered at least 6 hours or more in advance of the start of the prodromal phase or aura phase, preferably at least 12 hours or more in advance of the prodromal phase or aura phase, especially at least 24 hours, 48 hours, 72 hours or even longer in advance of the prodromal phase or aura phase.

The administration of an effective amount of an antiestrogen to male migraine sufferers daily or every other day on a continuing basis for a period of time that is sufficiently far in advance of the onset of the prodromal phase or aura phase prevents the prodromal phase or aura phase from developing in the first place. As a result, the present invention prevents the development not only of the acute headache phase, but also of the undesirable mood changes, photophobia and phonophobia and other neurologic disturbances that characterize the prodromal and aura phases.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

What is claimed is:

1. A method of preventing the occurrence of migraine headaches in a patient who is a previous sufferer of migraine headaches, said method comprising administering an effective amount therefor of an antiestrogen to said patient for a period of time and sufficiently far in advance of the occurrence of a migraine headache to prevent the development of the prodromal phase and/or aura phase of the migraine headache.

2. The method according to claim 1, wherein the antiestrogen is clomiphene or a pharmaceutically acceptable salt thereof.

3. The method according to claim 2, wherein the antiestrogen is clomiphene.

4. The method according to claim 2, wherein the antiestrogen is clomiphene citrate.

5. The method according to claim 4, wherein the antiestrogen is a clomiphene citrate composition comprising a content of clomiphene citrate trans isomer and optionally a content of clomiphene citrate cis isomer, wherein the content of the trans isomer ranges from 71-100% by weight of the total weight of trans isomer+cis isomer in the composition and the content of the cis isomer ranges from 0-29% by weight of the total weight of trans isomer+cis isomer in the composition.

6. The method according to claim 1, wherein the antiestrogen is tamoxifen or a pharmaceutically acceptable salt thereof.

7. The method according to claim 6, wherein the antiestrogen is tamoxifen.

8. The method according to claim 6, wherein the antiestrogen is tamoxifen citrate.

9. The method according to claim 1, wherein the antiestrogen is toremifene or a pharmaceutically acceptable salt thereof.

10. The method according to claim 9, wherein the antiestrogen is toremifene.

11. The method according to claim 9, wherein the antiestrogen is toremifene citrate.

12. The method according to claim 1, wherein the patient is a man.

13. The method according to claim 12, wherein the man is androgen deficient and/or menopausal.

14. The method according to claim 12, wherein the man is at least 50 years of age.

15. The method according to claim 1, wherein the antiestrogen is administered at least 6 hours in advance of the prodromal phase.

16. The method according to claim 15, wherein the antiestrogen is administered at least 24 hours in advance of the prodromal phase.

17. The method according to claim 1, wherein the antiestrogen is administered daily for a period of at least two days.

18. The method according to claim 17, wherein the antiestrogen is administered daily for a period of at least one week.

19. The method according to claim 18, wherein the antiestrogen is administered daily for a period of at least one month.

20. The method according to claim 19, wherein the antiestrogen is administered daily for a period of at least one year.

21. A method of preventing the onset of the prodromal phase of a migraine headache in a patient susceptible to suffering migraine headaches, said method comprising administering an effective amount therefor of an antiestrogen to the patient for a period of time and sufficiently far in advance of the onset of the prodromal phase or aura phase to prevent the onset of the prodromal phase or aura phase from occurring.

22. The method according to claim 21, wherein the patient is a man.

23. The method according to claim 22, wherein the man is androgen deficient and/or menopausal.

24. The method according to claim 22, wherein the man is at least 50 years of age.

25. The method according to claim 21, wherein the antiestrogen is clomiphene.

26. The method according to claim 21, wherein the antiestrogen is clomiphene citrate trans isomer.

27. The method according to claim 26, wherein the antiestrogen is a clomiphene citrate composition comprising a content of clomiphene citrate trans isomer and optionally a content of clomiphene citrate cis isomer, wherein the content of the trans isomer ranges from 71-100% by weight of the total weight of trans isomer+cis isomer in the composition and the content of the cis isomer ranges from 0-29% by weight of the total weight of trans isomer+cis isomer in the composition.
